(19) United States
(12) Patent Application Publication
(10) Pub. No.: US 2016/0175057 A1
(43) Pub. Date: Jun. 23, 2016

(54) ASSISTANCE DEVICE FOR IMAGING SUPPORT OF A SURGEON DURING A SURGICAL OPERATION

(71) Applicant: MAQUET GMBH, RASTATT (DE)

(72) Inventors: Bastian Ibach, KARLSRUHE (DE); MICHAEL BERNHART, KARLSRUHE (DE)

(73) Assignee: MAQUET GMBH, RASTATT (DE)

(21) Appl. No.: 15/054,743

(22) Filed: Feb. 26, 2016

Related U.S. Application Data
(63) Continuation-in-part of application No. PCT/EP2014/068585, filed on Sep. 2, 2014.

(30) Foreign Application Priority Data
Sep. 5, 2013 (DE) 10 2013 109 677.8

(51) Int. Cl. A61B 34/20; A61B 1/06; A61B 1/04; A61B 1/313; A61B 1/00; A61B 17/34

(52) U.S. Cl. CPC A61B 34/20; A61B 1/00045; A61B 17/3423; A61B 1/04; A61B 1/00009; A61B 1/3132; A61B 1/00006; A61B 1/00016; A61B 1/0661; A61B 1/00066; A61B 2034/301

(57) ABSTRACT

A surgical device is disclosed. The surgical device has an endoscope having a camera that generates image data, a viewing device that displays a moving image based on image data generated by the camera, and a manipulator that is coupled with the endoscope, the endoscope being movable by the manipulator. The surgical device also has a controller that controls the manipulator based on a control command, the moving image displayed on the viewing device changing based on a movement of the endoscope. The surgical device further has a sensor coupled with the controller, and an operating element coupled with the controller, the operating element having a first operating state and a second operating state. The sensor detects a moving object, which is used in a surgical procedure, and generates a movement signal based on a movement of the moving object.

### CROSS REFERENCE TO RELATED APPLICATIONS

The present application is a continuation-in-part filed under 35 U.S.C. §111(a), and claims the benefit under 35 U.S.C. §§365(c) and 371 of PCT International Application No. PCT/EP2014/068585, filed Sep. 2, 2014, and which designates the United States of America, and German Patent Application No. 10 2013 109 677.8, filed Sep. 5, 2013. The disclosures of these applications are herein incorporated by reference in their entirety.

### TECHNICAL FIELD

The present disclosure is directed to an assistance device for imaging support of a surgeon during a surgical operation, comprising an endoscope with a camera for generation of image data, a viewing device for presenting a moving image on the basis of the image data generated by the camera, a manipulator coupled with the endoscope for moving the endoscope, and a control unit for optional actuating of the manipulator in dependence on a control command, such that the displayed moving image on the viewing device can be influenced by moving the endoscope.

### BACKGROUND

During a laparoscopic procedure, a surgeon typically looks at a moving image of the operation site on a viewer, such as a monitor. In the moving image, one can see the laparoscopic instrument with which the surgeon is manipulating the anatomical structures and organs in the patient. The moving image presented on the viewer in real time is recorded by a camera, which is part of an endoscope introduced into the body of the patient via a trocar and directed to the operation site.

The endoscope is usually held by an assistant, who stands alongside the surgeon during the operation. The assistant tries to direct the endoscope at the operation site so that the target region in which the tips of the instruments and the anatomical structures being manipulated can be seen is located in a reference position of the moving image. Usually this reference position lies roughly in the middle of the moving image. If the image section being viewed is to be changed, the endoscope may be moved in order to bring the new target region again into the middle of the moving image.

Various image movements are relevant to the surgeon looking at the viewer. There are two-dimensional changes in the image section, for example, movements of the image section directed upward and downward on the viewer, movements of the image section directed left and right on the viewer, as well as combined movements, such as from bottom left to top right. Moreover, the image section on the viewer may also be changed by a corresponding zoom operation in the third dimension, e.g., enlarged or reduced.

Assistance devices for imaging support of a surgeon that do not utilize a human assistant are known from the prior art. Accordingly, these assistance devices may be able to be operated by the surgeon himself through corresponding control commands.

For example, an operating concept is known from the prior art in which the surgeon can dictate the direction of movement of the image presented on the viewer by a head movement. Using an activation pedal, the endoscope movement is enabled. For this, reference is made for example to the publications EP 2169348 B8, EP 2052675 A1, US 2009/0112056 A1, EP 0761177 B1 and U.S. Pat. No. 5,766,126 A.

Other operating concepts involve a voice control or a control of the endoscope movement via a foot pedal outfitted with several switches. For this, reference is made to the publications U.S. Pat. No. 6,932,089 B1, US 2006/0100501 A1 and US 2011/0257475 A.

Assistance devices which enable an automatic tracking of marked instruments are described in publications U.S. Pat. No. 6,820,545 A1 and DE 19529950 C1.

Publication EP 1937177 B1 proposes an operating lever also known as a joystick for the operation of an assistance device, which is arranged on a laparoscopic instrument. The surgeon can thus also control the endoscope movement with his hand holding the instrument.

Finally, a fully automatic system which enables an automatic tracking of marked instruments is described in DE 199 61 971 B4.

Despite the above technical solutions, there still exists a desire to optimize an assistance device so that it enables suitably complete automation of the endoscope tracking so that the surgeon is burdened as little as possible with the operation of the assistance device, while allowing for suitable outcomes for patients. Furthermore, the operation of such an assistance device should be easy to learn and easy to carry out.

### SUMMARY OF THE DISCLOSURE

A problem which the present disclosure addresses is to indicate an assistance device which the surgeon can operate easily and intuitively during a surgical operation.

The present disclosure may solve this problem in an assistance device of the above mentioned kind via a sensor unit coupled with the control unit, which may detect a moving object for the performance of the surgical procedure and may generate a movement signal corresponding to the object's movement, on the basis of which the control unit generates the control command. The problem may also be addressed via an operating element coupled to the control unit, which can be activated by the surgeon to set an enable state in which the actuating of the manipulator is enabled by the control unit for the moving of the endoscope.

The invention may provide a suitable interaction of an independently functioning sensor unit, e.g., without action by the surgeon, and an operating element explicitly activated by the surgeon. The operating element may be used (e.g., solely) to set an enable state in which the sensor unit is switched to an active state and independently detects a moving object for the performance of the surgical procedure and generates a movement signal corresponding to the object's movement (e.g., forming the basis for the actuating of the manipulator by the control unit and thus the tracking of the endoscope). In this way, by activating the operating element the surgeon may tell the assistance device that he wishes a tracking of the endoscope at a given time, whereupon the sensor unit and the control unit coupled to it take over the control of the endoscope tracking. With this, it is possible for the surgeon to use the assistance device for imaging support during the surgical procedure in a simple and reliable manner.

The sensor unit according to the present disclosure may form a separate unit from the endoscope camera. For example, it may be designed to detect an object situated outside the human body that is moved during the surgical procedure and to make use of the detected object's movement for the actuating of the manipulator. The moving object can be, for example, a part of a surgical instrument located outside the patient's body, which is introduced into the patient's body by a trocar tube.

The sensor unit can be used to detect the movement of a surgical instrument forming the moving object relative to a reference object. The reference object may be, for example, the trocar tube by which the instrument is introduced into the patient's body.

The control command may be provided for the actuating of the manipulator which is generated by the control unit on the basis of the movement signal, which is generated by the sensor unit with the detecting of the movement of the surgical instrument relative to the reference object. The control command may include a zoom command, which produces a zooming movement of the camera by the manipulator. In this embodiment, for example, the movement of the surgical instrument along an axis defined by the reference object, such as for example the longitudinal axis of the trocar tube, may be detected and this unidimensional object movement may be converted into a corresponding control command, via which the endoscope is moved along the optical axis of the camera optics contained therein (e.g., in order to perform a corresponding zoom operation).

If the sensor unit detects the movement of the surgical instrument relative to a trocar tube, forming the reference object, the movement signal generated by the sensor unit may indicate the movement of the surgical instrument relative to an entry point at which the trocar tube enters the body of the patient being treated. This entry point may form a largely (e.g., substantially) fixed reference point, which can be used in determining the object's movement.

For example, the sensor unit arranged in the trocar tube may comprise a light source and an image sensor, which are oriented to a window formed in an inner wall of the trocar tube, as well as a processor. The image sensor may take pictures (e.g., in succession) of the surgical instrument moving past the window of the trocar tube and illuminated by the light source and the processor may generate the movement signal due to differences in the consecutively taken pictures. The movement signal in this embodiment may indicate the position of the surgical instrument relative to the trocar tube along its tube axis. This instrument position relative to the trocar tube can be used as zoom information in order to move the endoscope situated in the patient's body along the optical axis of the camera optics contained in the endoscope and thus reduce or enlarge the image feature shown on the viewing device.

The sensor unit may be arranged in an enlarged instrument entrance of the trocar tube. If there is a check valve present in the instrument entrance of the trocar tube, which prevents an escaping of gas blown into the body of the patient, the sensor unit may be arranged in front of the check valve. In this way, traditional trocar tubes can be retrofitted with the sensor unit in a relatively simple manner. It may also be relatively easy to replace a defective sensor unit in this arrangement.

The control unit, in generating the control command in addition to the movement signal generated by the sensor unit, may also account for the image data generated by the camera. Thus, the control unit may contain an image processing module, which on the basis of the image data generated by the camera, may detect a surgical instrument as the moving object in the displayed moving image and may determine a position deviation of the surgical instrument relative to a fixed reference position within the displayed moving image. The control unit may then determine, in the enable state via the position deviation, a nominal value to be factored into the control command. The control unit may also then actuate the manipulator in dependence on this nominal value such that the surgical instrument detected in the displayed moving image is brought into the reference position that is determined by tracking of the endoscope.

For example, two-dimensional changes in the image feature on the viewing device, such as movements of the image feature to the top and bottom or to the right and left may be carried out by way of the instrument recognition performed inside the patient's body. An enlargement or reduction of the image feature on the viewing device by a zoom operation can be performed in addition by the movement signal which the sensor unit may generate outside the patient's body. In this way, the movement signal generated by the sensor unit and the image data generated by the camera may be combined in especially advantageous manner to produce the desired image movements on the viewing device.

For the instrument recognition, the image processing module may detect the tip of the medical instrument and may determine the position deviation of this instrument tip. The instrument recognition performed by the image processing module, which may furnish information about the position of the instrument tip in the moving image, can be combined with the functioning of the operating element, which may act as an enable switch. For example, it is possible to move the moving image with the instrument tip dynamically in any given direction. The moving image may follow the identified instrument for as long as the enable state is present. For example, when (e.g., as soon as) the enable state is ended, the moving image may stand (e.g., remain) still. Thus, in addition to allowing movements in fixed directions, such as up or down, or right or left, substantially any given direction of movement can be realized.

For example, the moving object detected by the sensor unit may be formed by a marking body, which can be placed on a surgical instrument or on the surgeon. In this way, the moving object used as reference for the endoscope tracking may be relatively simple to replace, for example by removing the marking body from one instrument and placing it on another instrument.

Preferably, the marking body may be a rigid body, having at least three non-collinear marking points which the sensor unit can detect. Because the marking points may not lie on the same line in space, they may define a marking plane whose movement in space can be detected by the sensor unit.

In an alternative embodiment, the sensor unit may contain an acceleration sensor, e.g., a three-axis acceleration sensor, which may detect the moving object in space. This acceleration sensor can be placed, for example, on a bracelet which the surgeon wears in the region of his wrist. Alternatively, it can also be affixed to the back of the surgeon's hand. The acceleration sensor may also be disposed on the surgical instrument.

The sensor unit may utilize and/or work with any suitable measurement principle for the detection of the moving object. Thus, the sensor unit can contain, for example, an optical sensor, as indicated above. Also, for example, a magnetically operating sensor, such as a differential transformer (e.g., linear variable differential transformer or LVDT) or an electromechanically operating sensor, may be used. Such an electromechanical sensor can be designed, for example, to pick up the movement of the object by a roller and transform the roller movement into an electrical measurement quantity, which then represents the movement signal. RFID sensors may also be used to detect the moving object, the RFID sensor being formed for example by a transponder arranged on the object and a reading device communicating with this transponder. The sensor unit can also work, for example, by an electromagnetic tracking method.

The assistance device may include wireless transmission of the movement signal generated by the sensor unit to the control unit. For example, the transmission of the movement signal can occur by radio. Also, for example, wire-line signal transmission may be used.

The operating element (e.g., operating unit) may be formed by a single switch element with two switching states, of which one switching state is assigned to the enabled state and the other switching state to a disabled state, in which the actuating of the manipulator by the control unit is blocked. For example, the switch element may have precisely two switching states. Because the surgeon in this case may only operate a single switch element, the handling may be significantly simplified. Thus, the movement signal generated by the sensor unit may only take effect when the surgeon activates the switch element and thus enables the movement of the manipulator. The single switch element can be easily and distinctly positioned, for example on the surgical instrument with which the surgeon is performing the operation, or on the hand or fingers of the surgeon. The switch element can also be designed as a pedal switch.

Since (e.g., only) a single switch element may be provided to the surgeon, by which he can control the assistance device, the operation of the assistance device may be simple and intuitive.

### BRIEF DESCRIPTION OF THE DRAWINGS

The invention shall now be explained more closely with the help of the figures:

FIG. 1 illustrates a block diagram of an exemplary assistance device;

FIG. 2 illustrates an exemplary surgical instrument and an exemplary trocar tube in which a sensor unit is accommodated;

FIG. 3 illustrates the makeup of an exemplary sensor unit accommodated in the trocar tube according to FIG. 2;

FIG. 4 illustrates an alternative embodiment of the sensor unit, which may detect a marking body arranged on a surgical instrument; and

FIG. 5 illustrates a schematic representation to illustrate an alternative placement of the marking body.

### DETAILED DESCRIPTION AND INDUSTRIAL APPLICABILITY

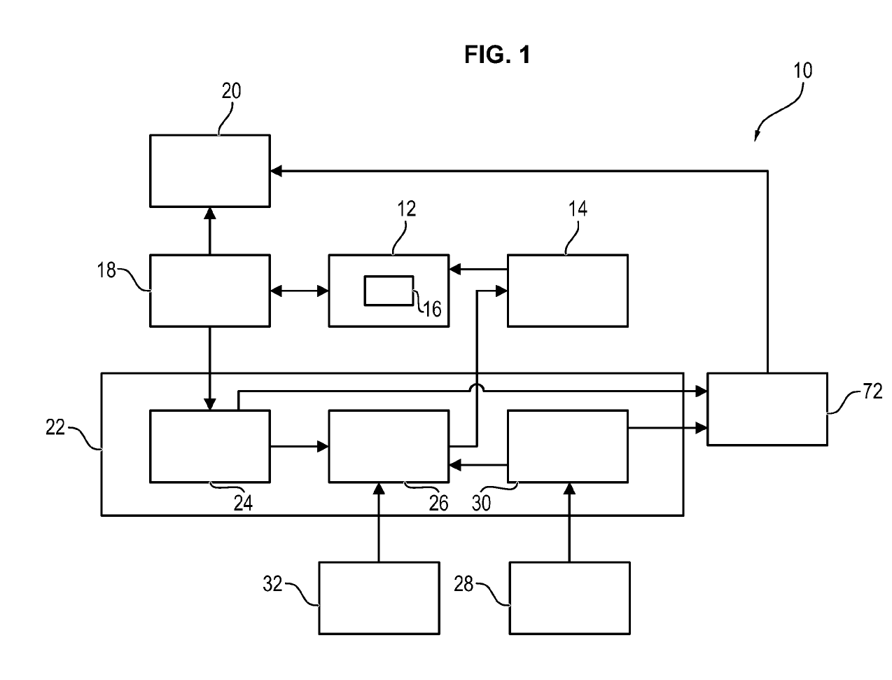

FIG. 1 shows an exemplary assistance device in a block diagram.

The assistance device 10 may comprise an endoscope 12, which may be held by a manipulator 14, configured for example as a robot arm. The manipulator 14 may have mechanical degrees of freedom enabling a tracking of the endoscope 12. The endoscope 12 may be movable by the manipulator 14.

A camera 16 may be disposed on the endoscope 12, which may form a unit with the endoscope 12. Camera 16 may also, for example, be integrated in the endoscope 12 (e.g., from the outset). The camera 16 may record an image of the anatomical structure being treated. Accordingly, camera 16 may generate image data which is put out (e.g., provided) in the form of a data stream to a camera controller 18. The camera controller 18 may relay this image data to a viewing device 20, such as a monitor screen, on which a moving image of the anatomical structure being treated is displayed according to the image data.

The camera controller 18 may relay the image data stream via an image detection module, such as for example a frame grabber, to a control unit 22 (e.g., a controller). The control unit 22 may contain an image processing module 24, which uses the image data stream supplied to it as an input signal in order to carry out an instrument recognition. The instrument recognition may operate, for example, by making use of image processing algorithms. In this process, surgical instruments visible in the moving image may be recognized by the image processing module 24 and their positions may be detected. In particular, the image processing module 24 may determine a position deviation for a given recognized instrument, which the tip of this instrument may have relative to the mid point of the moving image displayed on the viewing device 20 (e.g., monitor screen).

The image processing module 24 may put out the determined position deviations of the instrument tips to a path control 26, which may determine from this nominal values for the actuation of the manipulator 14. If appropriate, the manipulator 14 may be actuated based on these nominal values to move the endoscope 12 so that the instrument tip of an instrument selected as a guidance instrument may be brought to the middle of the moving image.

The assistance device 10 may have an operating element 28, which may be coupled to an interface control 30 contained in the control unit 22. The operating element 28 may be a monostable push button, activated for example by pressing, with two switching states (e.g., precisely two switching states), for example, an activated state and a non-activated state. For example, the precisely two switching states may be a first operating state (e.g., an enable state) and a second operating state (e.g., a disable state).

Moreover, the assistance device 10 may contain a graphic user interface 72, which may be coupled to the image processing module 24 and interface control 30, or may be coupled to the viewing device 20 (e.g., monitor screen).

Finally, the assistance device 10 may comprise a sensor unit 32 (e.g., a sensor), which may be connected to the path control 26 of the control unit 22.

The sensor unit 32 may detect an object, which may be moved outside the body of the patient during the performance of the surgical procedure (e.g., to generate a movement signal representing the motion of this object). The sensor unit 32 may put out the movement signal to the path control 26. The connection of the sensor unit 32 to the path control 26 can occur via a wire connection or also wirelessly (for example, by radio).

The path control 26 may control the manipulator 14 based on the nominal values generated in the course of the instrument recognition (e.g., which may be generated from the position deviations put out (e.g., provided) by the image processing module 24), and/or may control the manipulator 14 based on the movement signal generated by the sensor unit 32. For example, the nominal values and the movement signal may be combined by the path control 26 into a control command with which the path control 26 may control the manipulator 14 for the tracking of the endoscope 12.

Using the operating element 28, the surgeon can set an enable state (e.g., a first operating state) and a disable state (e.g., a second operating state) of the assistance device 10. The enable state may be associated with the activated switching state of the operating element 28, and the disable state may be associated with the non-activated switching state of the operating element 28. For example, in the enable state an actuation of the manipulator 14 may occur based on the control command generated by the path control 26. Also for example, if the disable state is set, there may be no actuation of the manipulator 14 by the control command.

In the exemplary embodiment shown in FIG. 1, the actuation of the manipulator 14 may occur such that the nominal values obtained from the instrument recognition, which may be included in the control command put out (e.g., provided) by the path control 26 to the manipulator 14, are used for the movement of the endoscope 12 in a plane perpendicular to the optical axis of the camera optics. The movement signal generated by the sensor unit 32, which may be included in the control command, may be used for a zoom movement of the endoscope 12 along the optical axis of the camera optics. Thus, there may be an actuation of the manipulator unit 14, for example, both based on control data which is generated in the patient's body and based on control data which is obtained outside of the patient's body.

Also, for example, the control command may be generated (e.g., solely generated) from the movement signal generated from the sensor unit 32.

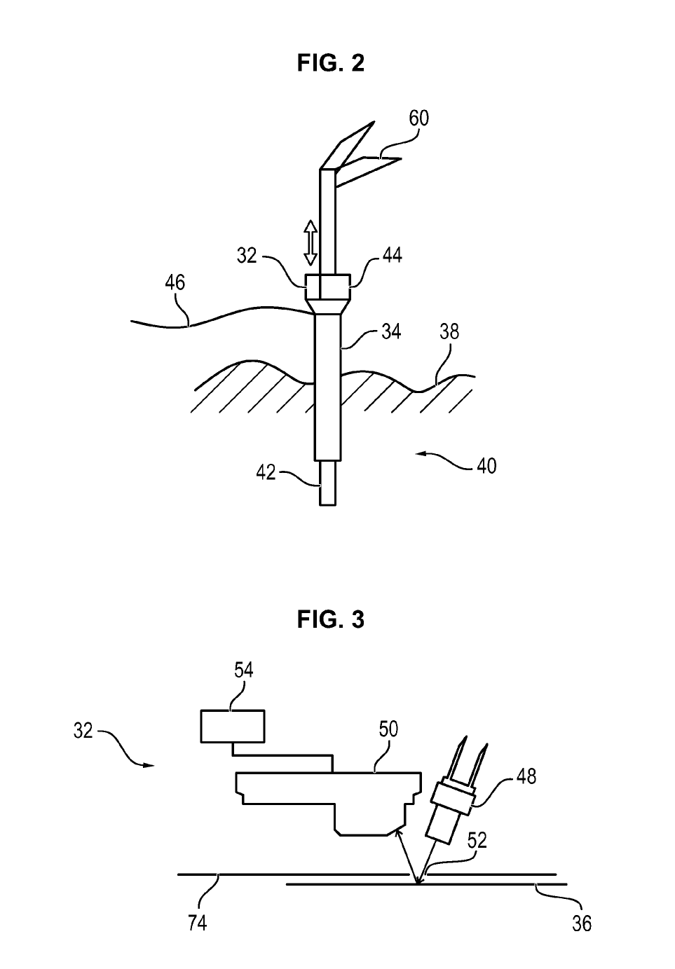

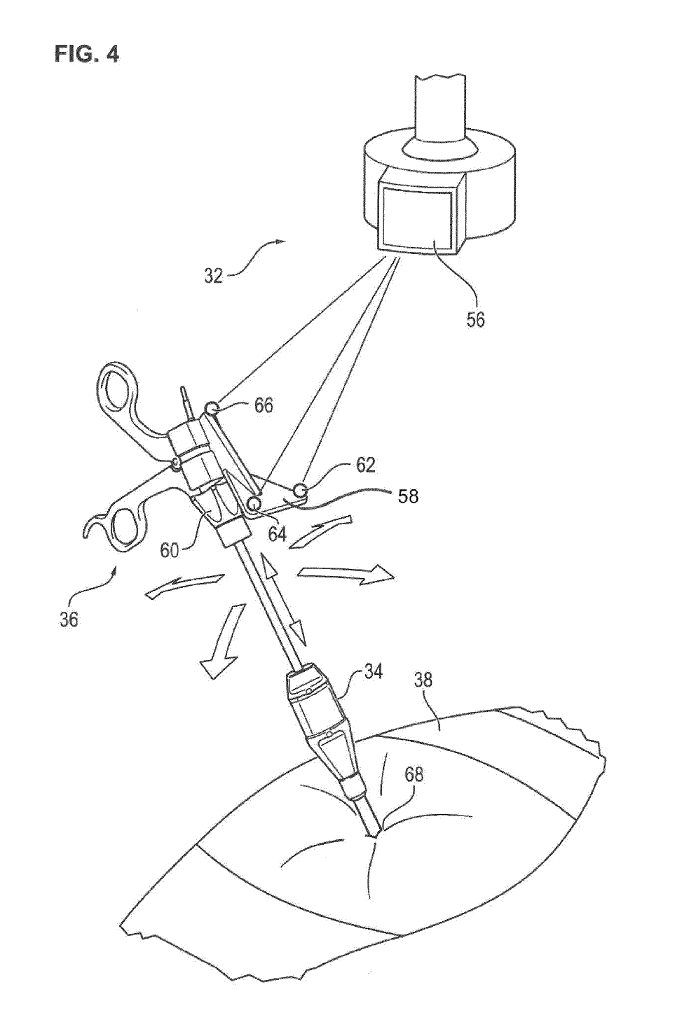

FIGS. 2 and 3 show an exemplary embodiment of the sensor unit 32.

In this embodiment, the sensor unit 32 may be integrated in a trocar tube 34, which may serve to introduce a surgical instrument 36 through an abdominal wall 38 into an abdominal cavity 40. The surgical instrument 36 may be inserted by its tip 42 into an enlarged instrument entrance 44 of the trocar tube 34 and shoved (e.g., inserted) relatively far (e.g., deeply) into the trocar tube 34 so that the instrument tip 42 emerges from the trocar tube 34 and is exposed in the abdominal cavity 40.

The sensor unit 32 may be arranged in the instrument entrance 44 of the trocar tube 34. The sensor unit 32 may serve to detect the movement of the surgical instrument 36 relative to the trocar tube 34 along its tube axis and may transmit the movement signal corresponding to this relative movement to the path control 26. Based on the movement signal, the path control 26 may generate the control command for the actuating of the manipulator 14, while the movement signal generated by the sensor unit 32 may cause a zoom movement of the endoscope 12 along the optical axis of the camera optics. Accordingly, the longitudinal axis of the trocar tube 34 and the optical axis of the camera optics may coincide. For the transmission of the movement signal, a transmission line 46 may be provided (e.g., as illustrated in FIG. 2), which connects the sensor unit 32 to the path control 26 of the control unit 22.

FIG. 3 shows an exemplary layout of the sensor unit 32 integrated in the trocar tube 34.

The sensor unit 32 may comprise a semiconductor laser 48 as the light source and an image sensor 50, which are arranged in the region of a window 52 in the instrument entrance 44 of the trocar tube 34, which may be disposed in the inner wall 74 of the instrument entrance 44.

For example, the semiconductor laser 48 may be a surface emitter (e.g., VCSEL, or vertical-cavity surface-emitting laser). For example, semiconductor laser 48 may be a semiconductor chip in which light is emitted perpendicular to the chip plane.

The semiconductor laser 48 and the image sensor 50 (e.g., coordinated with the semiconductor laser 48) may be oriented toward the window 52 such that the part of the surgical instrument 36 moving past the window 52 and illuminated by the semiconductor laser 48 may be projected onto the image sensor 50. The image sensor 50 may thus, for example, take successive pictures of the surgical instrument 36 moving past the window 52.

The sensor unit 32 may have a microprocessor 54, coupled with the image sensor 50, which may evaluate the pictures taken successively by the sensor 50. The microprocessor 54 may detect differences in the successively taken pictures of the surgical instrument moving past the window 52 and may generate the movement signal with the aid of these differences.

FIG. 4 illustrates another exemplary embodiment of the sensor unit 32. For example, the sensor unit 32 may comprise a 3D camera 56, for example a camera which may detect the movement of an object and generate a movement signal corresponding to this object's movement.

For example, as illustrated in FIG. 4, a marking body 58 may be provided, which can be placed on a handle 60 of the surgical instrument 36. The marking body 58 may be made, for example, from a rigid plastic. In the present embodiment, the marking body 58 may have a plurality of (e.g., three) marking points 62, 64, and 66, which may not be arranged or disposed collinearly (e.g., not on a straight line). Based on the plurality of (e.g., three) marking points 62, 64 and 66, the 3D camera 56 can thus detect the arrangement of the marking body 58 in space and thus that of the instrument 36, and may generate the movement signal from this. The movement of the marking body 58 may be referred (e.g., related to) to an entry point 68, which may be formed by the point at which the trocar tube 34 enters the abdominal wall 38.

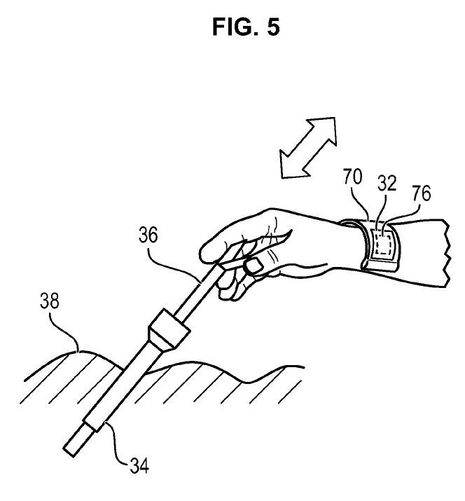

FIG. 5 illustrates another exemplary embodiment. For example, as illustrated in FIG. 5, a bracelet 70 may be provided, which the surgeon may wear on his or her wrist. The bracelet 70 may contain a three-axis acceleration sensor 76, which may detect the movement of the surgeon's wrist in space and may send out a corresponding movement signal. This movement signal may be sent wirelessly (e.g., by radio) to the path control 26. Also, for example, the acceleration sensor 76 can also be arranged on the back of the surgeon's hand or on the surgical instrument 36.

It will be apparent to those skilled in the art that various modifications and variations can be made to the disclosed method and apparatus. Other embodiments will be apparent to those skilled in the art from consideration of the specification and practice of the disclosed method and apparatus. It is intended that the specification and the disclosed examples be considered as exemplary only, with a true scope being indicated by the following claims.

What is claimed is:

1. A surgical device, comprising:
an endoscope having a camera that generates image data;
a viewing device that displays a moving image based on image data generated by the camera;
a manipulator that is coupled with the endoscope, the endoscope being movable by the manipulator;
a controller that controls the manipulator based on a control command, the moving image displayed on the viewing device changing based on a movement of the endoscope;
a sensor coupled with the controller; and
an operating element coupled with the controller, the operating element having a first operating state and a second operating state;
wherein the sensor detects a moving object, which is used in a surgical procedure, and generates a movement signal based on a movement of the moving object;
wherein the controller generates a control command based on the movement signal;
wherein in the first operating state, control of the manipulator by the controller to move the endoscope is enabled; and
wherein in the second operating state, control of the manipulator by the controller to move the endoscope is disabled.

2. The surgical device according to claim 1, wherein the moving object is a surgical instrument, and the sensor detects the movement of the surgical instrument relative to a reference object.

3. The surgical device according to claim 1, wherein the control command includes a zoom command, the manipulator being controlled via the zoom command to make a zooming movement with the camera.

4. The surgical device according to claim 2, wherein:
the sensor detects the movement of the surgical instrument relative to a trocar tube in which the surgical instrument is guided; and
the trocar tube is the reference object.

5. The surgical device according to claim 1, wherein the controller generates the control command based on the movement signal generated by the sensor and the image data generated by the camera.

6. The surgical device according to claim 1, wherein:
the moving object is a surgical instrument;
the controller includes an image processing module that detects the surgical instrument in the displayed moving image based on the image data generated by the camera;
the image processing module determines a position deviation of the surgical instrument relative to a reference position established within the displayed moving image; and
in the enable state, the controller determines a nominal value to be factored into the control command based on the position deviation, and the controller actuates the manipulator based on this nominal value so that the surgical instrument detected in the displayed moving image is brought into the determined reference position by tracking of the endoscope with the camera.

7. The surgical device according to claim 1, wherein the movement signal generated by the sensor is transmitted to the controller via wireless transmission.

8. The surgical device according to claim 1, wherein the operating element is a single switch element having precisely two switching states, the precisely two switching states being the first operating state and the second operating state.

9. A surgical device, comprising:
an endoscope having a camera that generates image data;
a viewing device that displays a moving image based on image data generated by the camera;
a manipulator that is coupled with the endoscope, the endoscope being movable by the manipulator;
a controller that controls the manipulator based on a control command, the moving image displayed on the viewing device changing based on a movement of the endoscope;
a sensor coupled with the controller, the sensor including one of a surface-emitting laser, a plurality of marking points, or an acceleration sensor; and
an operating element coupled with the controller, the operating element having a first operating state and a second operating state;
wherein the sensor detects a moving object, which is used in a surgical procedure, and generates a movement signal based on a movement of the moving object;
wherein the controller generates a control command based on the movement signal; and
wherein in the first operating state, control of the manipulator by the controller to move the endoscope is enabled.

10. The surgical device according to claim 9, wherein in the second operating state, control of the manipulator by the controller to move the endoscope is disabled.

11. The surgical device according to claim 9, wherein the moving object is a surgical instrument, and the sensor detects the movement of the surgical instrument relative to a reference object.

12. The surgical device according to claim 11, wherein:
the sensor detects the movement of the surgical instrument relative to a trocar tube in which the surgical instrument is guided; and
the trocar tube is the reference object.

13. The surgical device according to claim 12, wherein:
the sensor is disposed in the trocar tube and includes a light source and an image sensor, which are oriented to a window formed in an inner wall of the trocar tube, and a processor; and
the image sensor takes pictures, in succession, of the surgical instrument moving past the window of the trocar tube and illuminated by the light source.

14. The surgical device according to claim 13, wherein:
the processor generates the movement signal based on differences in the consecutively taken pictures; and
the sensor is disposed in an enlarged instrument entrance of the trocar tube.

15. The surgical device according to claim 9, wherein:
the moving object is a marking body that is disposed on a surgical instrument; and
the marking body is a rigid body having at least three non-collinear marking points that are detectable by the sensor.

16. The surgical device according to claim 9, wherein:
the sensor includes the acceleration sensor; and
the acceleration sensor is disposed on a bracelet.

17. A system, comprising:
an endoscope having a camera that generates image data;
a monitor screen that displays a moving image based on image data generated by the camera;
a surgical robot arm that is coupled with the endoscope, the endoscope being movable by the surgical robot arm;
a controller that controls the surgical robot arm based on a control command, the moving image displayed on the monitor screen changing based on a movement of the endoscope;
a sensor coupled with the controller; and
an operating element coupled with the controller, the operating element having a first operating state and a second operating state;
wherein the sensor detects a surgical instrument, and generates a movement signal based on a movement of the surgical instrument;
wherein the controller generates a control command based on the movement signal;
wherein in the first operating state, control of the surgical robot arm by the controller to move the endoscope is enabled; and
wherein in the second operating state, control of the surgical robot arm by the controller to move the endoscope is disabled.

18. The system according to claim 17, wherein the operating element is a monostable push button.

19. The system according to claim 17, wherein the operating element is a single switch element having precisely two switching states, the precisely two switching states being the first operating state and the second operating state.

20. The system according to claim 19, wherein the sensor includes one of a surface-emitting laser, a plurality of marking points, or an acceleration sensor.
